(19) United States
(12) Patent Application Publication
(10) Pub. No.: US 2022/0039402 A1
(43) Pub. Date: Feb. 10, 2022

(54) Compositions and Methods for Enhancing Quality of Bread and Baked Goods

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/281,502

(22) PCT Filed: Nov. 26, 2019
(86) PCT No.: PCT/US2019/063258

§ 371 Date: Mar. 30, 2021

Related U.S. Application Data
(60) Provisional application No. 62/772,256, filed on Nov. 28, 2018.

(51) Int. Cl. A21D 8/04

(52) U.S. Cl. CPC A21D 8/047; A21D 8/045

(57) ABSTRACT

The subject invention provides compositions and methods for enhancing the quality of bread and other baked goods. By baking the bread or other baked goods with a yeast-based biopreservative composition, the subject invention provides methods for enhancing the taste, texture and shelf-life of these food products. In certain embodiments, the yeast-based preservative composition comprises Wickerhamomyces anomalus and/or a microbial growth by-product. The composition can be used in place of or in addition to traditional baker's yeast.

### CROSS-REFERENCE TO RELATED APPLICATION

This application claims priority to U.S. Provisional Patent Application No. 62/772,256, filed Nov. 28, 2018, which is incorporated herein by reference in its entirety.

### BACKGROUND OF THE INVENTION

Modernization of agricultural and food processing technologies has led to an increased need for methods of keeping consumable products fresh and safe throughout transport and shelf-life. Insects and/or pests, physical injury, enzymatic degradation, and/or microbial activity can all be the cause of degradation in the quality of food products, leading to food waste and potential health problems for consumers.

Microbial growth, in particular, can alter food products drastically by causing, e.g., changes in smell, taste, color and/or texture. Microorganisms can also lead to illnesses, such as food poisoning or allergic reactions, if a contaminated food item is consumed.

The shelf span of breads and other baked goods is commonly affected by the growth of molds. Several species of molds, often called “bread molds,” will grow on bread, many of which produce fuzzy blue, green and/or black splotches on the surface. Bread molds thrive on the sugar and carbohydrates in bread; spores land on the surface and their growth is fueled by these organic substances. It can take five to seven days or less for bread left in the open to grow visible mold.

The species of bread mold that will appear on a food item depends on the type of spores that are present in the environment. Common food molds include Alternaria, Botrytis, Cladosporium, Fusarium, Geotrichum, Manoscus, Monilia, Mortierella, Mucor, Neurospora, Oidium, Oosproa, Penicillium, and Rhizopus, with those typically found on bread and other baked goods largely belonging to Aspergillus, Cladosporium, Penicillium, and Rhizopus. A specific example is Rhizopus stolonifer, or black bread mold. Some of these molds are harmless, but some can be allergenic, can cause nausea and/or vomiting, and/or can produce toxic substances called mycotoxins.

Some prior art methods of preserving baked products include freezing and/or refrigeration, which can affect the texture and moisture content, and the use of special anti-microbial packaging. Additionally, in the case of mass-produced foodstuffs, manufacturers compensate for spoilage by adding chemical preservatives to products. However, chemical preservatives (e.g., sodium benzoate, potassium sorbate or calcium propionate), can sacrifice the quality, taste, and overall integrity of processed food, and can be harmful to consumers over long term exposure.

Bread and baked goods are essential to many diets and cultures worldwide. Nonetheless, these food products are quickly susceptible to microbial contamination if not preserved properly. Thus, improved compositions and methods are needed for safely and effectively preserving breads and baked goods.

### BRIEF SUMMARY OF THE INVENTION

The subject invention relates to enhancing the quality of food products, particularly breads and baked goods. Specifically, the subject invention provides preservative compositions and methods for extending the consumable life of food products, especially breads and baked goods, and for enhancing the safety of food products for consumption using microbes and/or their growth by-products. Advantageously, the microbe-based products and methods of the subject invention are environmentally-friendly, non-toxic and cost-effective.

In preferred embodiments, the subject invention provides biopreservative compositions comprising microorganisms and/or microbial growth by-products. The composition may comprise, for example, active or inactive cells, fermentation medium, and/or growth by-products. The growth by-products can be produced by the microorganism of the composition, and/or they can be added to the composition in a purified or unpurified form.

In specific embodiments, the microorganisms are yeasts. In one embodiment, the yeast is Wickerhamomyces anomalus. In one embodiment, W. anomalus is used in combination with baker's yeast, or Saccharomyces cerevisiae. The composition can be formulated to comprise active or dormant yeast cells.

In one embodiment, the microorganisms are bacteria, for example, Bacillus coagulans. In a specific embodiment, the composition comprises B. coagulans GBI-30 (BC-30).

Examples of growth by-products according to the subject invention include one or more of: biosurfactants, biopolymers, enzymes, acids, solvents, alcohols, proteins, peptides, lipids, carbohydrates, amino acids, nucleic acids and others. In one embodiment, the microbial growth by-products comprise ethyl acetate.

In one embodiment, the microbial growth by-products comprise one or more biosurfactants selected from glycolipids, e.g., rhamnolipids, sophorolipids, trehalose lipids, cellobiose lipids or mannosylerythritol lipids; lipopeptides, e.g., surfactin, iturin, fengycin, viscosin, arthrofactin or lichenysin; and phospholipids, e.g., cardiolipin. In a specific exemplary embodiment, the biosurfactants comprise a blend of a sophorolipid, a surfactin and/or a phospholipid.

Preferably, when biosurfactants are present, the concentration is about 0.001% to about 0.5%, or from about 0.01% to about 0.1% by weight.

In an exemplary embodiment, the composition comprises W. anomalus and, optionally, S. cerevisiae and/or BC30; ethyl acetate; and/or about 0.1% by weight of a biosurfactant blend comprising a glycolipid, a surfactin and/or a phospholipid.

The biopreservative composition can also comprise appropriate additives and/or carriers depending on its formulation and intended use.

In certain embodiments, methods of cultivating the biopreservative compositions are provided. The compositions can be obtained through cultivation processes ranging from small to large scale, including, for example, submerged cultivation/fermentation, solid state fermentation (SSF), and modifications, hybrids or combinations thereof.

In certain embodiments, methods are provided for enhancing the quality of a baked good, wherein a biopreservative composition of the subject invention is baked into the baked good. More specifically, the methods comprise forming a dough using, for example, flour, water or another liquid, and optionally, one or more of a sugar, salt, fat, oil, egg, milk or flavoring, wherein the biopreservative composition is incorporated into the dough. In certain embodiments, the biopreservative composition is added in place of, or in addition to, a fermenting and/or leavening agent (e.g., standard baker's yeast). The method can further comprise mixing, kneading, folding, proofing, fermenting, glazing, scoring, chilling, forming, topping, baking and/or processing the dough in any other way according to standard procedures and depending on what baked good is being produced.

The subject methods enhance the quality of baked goods by, for example, extending the consumable life of baked goods, reducing the risk of illness or harm due to microbial contaminants (e.g., bread molds), as well as improving the taste, texture and/or nutritional value of the product, compared with products baked using traditional ingredients. Advantageously, the methods can be scaled for home bakers to large-scale mass-producers of baked goods.

In some embodiments, the method prevents and/or controls undesirable microbial growth in and on the baked good, thus prolonging the consumable life of the baked good and preventing microbial alteration and/or decomposition thereof. The method can also be used to enhance the safety of the baked good for consumption, e.g., by preventing food poisoning or illness from molds and other pathogenic food-borne microorganisms.

In some embodiments, the method improves the taste and/or texture of the baked good. For example, in one embodiment, the subject method intensifies the “sour” flavor of sourdough bread. In another embodiment, the method increases the rise of bread dough, leading to improved texture through a lighter, fluffier, airier, and/or more elastic baked bread.

In some embodiments, the method comprises adding a health-promoting probiotic to the baked good to enhance the nutritional properties of the baked good. In certain embodiments, the health-promoting probiotic is BC30. In certain specific embodiments, the BC30 is in spore form.

In preferred embodiments, the baked good is a food product made of dough (e.g., comprising flour of wheat, maize, rice, oats, rye, legumes, nuts, seeds, or other cereal crops) that requires leavening (e.g., by yeast fermentation, sodium bicarbonate, baking powder and/or cream of tartar) and is then baked in an oven (or another source of heat). In preferred embodiments, the baked good is bread (e.g., a loaf, roll, muffin, biscuit, breadstick, bun, pita, or naan) or another product, such as pizza crusts, bagels, pretzels, doughnuts, cakes, cookies, pastries, pancakes, brownies, waffles, pies, tarts, puddings, and the like.

In some embodiments, the method can be used simultaneously with other standard methods of preservation. For example, the method can be used in combination with refrigeration, freezing and/or active packaging.

In some embodiments, the method can be used to reduce or eliminate the need for freezing or refrigerating baked goods to preserve them for longer than, for example, a week.

Advantageously, the compositions and methods of the subject invention can be effective for preserving food and preventing food-borne illnesses without negatively altering the taste, smell, appearance, texture and/or nutritional value of food products. In fact, the compositions and methods can be used to enhance such properties of the food products, thus improving the overall experience and value of consuming the products.

Advantageously, the present invention can be used without releasing large quantities of inorganic compounds into the environment. Additionally, in preferred embodiments, the compositions and methods utilize components that are toxicologically safe and that meet the requirements for “organic” status. Thus, the present invention can be used for enhancing the quality of baked goods as a “green” product.

### DETAILED DESCRIPTION

The subject invention relates to enhancing the quality of food products, particularly breads and baked goods. Specifically, the compositions and methods of the subject invention can be effective for preserving food and preventing food-borne illnesses without negatively altering the taste, smell, appearance, texture and/or nutritional value of food products. In fact, in preferred embodiments, the compositions and methods can be used to enhance the organoleptic and/or nutritional properties of food products.

In preferred embodiments, the subject invention provides biopreservative compositions comprising microorganisms and/or microbial growth by-products. The composition may comprise, for example, active or inactive cells, fermentation medium, and/or microbial growth by-products. The growth by-products can be produced by the microorganism of the composition, and/or they can be added to the composition in a purified or unpurified form. Methods of enhancing the quality of baked goods using the subject compositions are also provided.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, proteins, and/or other cellular components. The microbes may be intact or lysed. In some embodiments, the microbes are present, with substrate in or on which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, 1×10¹¹, 1×10¹², 1×10¹³ or more CFU per gram or ml of the composition. As used herein, a propagule is any portion of a microorganism from which a new and/or mature organism can develop, including but not limited to, cells, spores, conidia, hyphae, mycelia, cysts, buds and seeds.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, and/or non-nutrient growth enhancers. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, a “biofilm” is a complex aggregate of microorganisms, such as bacteria, wherein the cells can adhere to each other and/or to a surface using, for example, an extracellular polysaccharide matrix. The cells in biofilms are physiologically distinct from planktonic cells of the same organism, which are single cells that can float or swim in liquid medium.

As used herein, the term “consumable life” of a food product means the length of time a product is fit for consumption. Consumable life includes the length of time the food product is safe for consumption, e.g., able to be consumed by a subject without causing harm to the subject or making the subject ill, and the length of time the food product is palatable, e.g., has not lost characteristics such as nutritional value, taste, smell, texture or appearance that make the food product desirable for consumption.

As used herein, the term “control” used in reference to a microorganism or a pest means killing, disabling, immobilizing, or reducing population numbers of the microorganism or pest, or otherwise rendering the microorganism or pest substantially incapable of causing harm.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites include, but are not limited to, biopolymers, enzymes, toxins, acids, solvents, alcohols, proteins, peptides, amino acids, vitamins, minerals, microelements, and biosurfactants.

As used herein, a “pest” is any organism, other than a human, that is destructive, deleterious and/or detrimental to humans or human concerns (e.g., food safety). Pests may cause and/or carry agents that cause infections, infestations and/or disease. Pests may be single- or multi-cellular organisms, including but not limited to, viruses, fungi, bacteria, parasites, protozoa, arthropods and/or nematodes. In some embodiments, the pest is a fungus or bread mold.

As used herein, the term “preservative” means a substance or chemical that prevents undesirable microbial growth and/or undesirable chemical changes in a product, which can lead to decomposition of the product. In the context of food products, preservatives are also useful for preventing foodborne illnesses, decreasing microbial spoilage, and/or preserving fresh attributes and nutritional quality of the food. A “biopreservative” is a preservative derived from a living organism or from the earth.

As used herein, “prevention” means avoiding, delaying, forestalling, or minimizing the onset or progression of a particular occurrence or situation (e.g., contamination, illness). Prevention can include, but does not require, absolute or complete prevention, meaning the occurrence or situation may still develop at a later time than it would without preventative measures. Prevention can include reducing the severity of the onset of an occurrence or situation, and/or inhibiting the progression of the occurrence or situation to one that is more severe.

As used herein, the term “probiotic” refers to microorganisms, which, when administered in adequate amounts, confer a health benefit on the host.

As used herein, “reduces” means a negative alteration, and “increases” means a positive alteration, wherein the alteration is at least 0.001%, 0.01%, 0.1%, 1%, 5%, 10%, 15%, 20%, 25%, 30%, 35%, 45%, 50%, 55%, 60%, 65%, 70%, 75%, 80%, 85%, 90%, 95%, 99%, or 100%, inclusive of all values therebetween.

As used herein, “reference” means a standard or control condition.

As used herein, a “salt-tolerant” microbial strain is capable of growing in a sodium chloride concentration of fifteen (15) percent or greater. In a specific embodiment, “salt-tolerant” refers to the ability to grow in 150 g/L or more of NaCl.

As used herein, the term “spoilage” means the spoiling, deterioration and/or contamination of a food product to the point that it is inedible, or its quality for edibility becomes reduced. Food that is capable of spoilage is called “perishable food.”

As used herein, “surfactant” means a compound that lowers the surface tension (or interfacial tension) between two liquids or between a liquid and a solid. Surfactants act as, e.g., detergents, wetting agents, emulsifiers, foaming agents, and dispersants. A biosurfactant is a surfactant produced by a living cell, e.g., a microbe.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example, within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein

Other features and advantages of the invention will be apparent from the following description of the preferred embodiments thereof, and from the claims. All references cited herein are hereby incorporated by reference.

### Compositions According to the Subject Invention

In one embodiment, the subject invention provides microbe-based biopreservative compositions comprising a microorganism and/or a microbial growth by-product.

The biopreservative compositions can be obtained through cultivation processes ranging from small to large scale. These cultivation processes include, but are not limited to, submerged cultivation/fermentation, solid state fermentation (SSF), and modifications, hybrids or combinations thereof. Thus, in certain embodiments, the composition can comprise microbial cells, residual fermentation medium in which the microorganism was cultivated, and/or one or more microbial growth by-products.

In preferred embodiments, the microorganism is a yeast. In one embodiment, the yeast is Wickerhamomyces anomalus.

In certain embodiments, the composition comprises W. anomalus in combination with one or more additional microorganisms. For example, in one embodiment, the composition comprises W. anomalus and baker's yeast, or Saccharomyces cerevisiae.

The total amount of yeast present in the composition can be at least 1×10³, and as much as 1×10¹² CFU/ml, or 1×10⁵to 1×10¹⁰ CFU/ml, or 1×10⁷ to 1×10⁹CFU/ml.

In some embodiments, the composition comprises a bacterium, such as a probiotic. For example, in one embodiment, the composition comprises Bacillus coagulans GBI-30, or BC30, at an amount of 1×10⁸ to 1×10¹² CFU/ml, or 1×10⁹ to 1×10¹¹ CFU/ml. In preferred embodiments, the BC30 is in spore form.

In one embodiment, the microorganism(s) of the biopreservative composition can be active or inactive. In certain embodiments, the microorganisms are dormant or in spore form.

The microbial growth by-products of the subject composition can be produced by the microorganism(s) of the composition, and/or they can be added in addition to any metabolites produced by the microorganism(s). Examples of growth by-products according to the subject invention include biosurfactants, biopolymers, enzymes, acids, solvents, alcohols, proteins, peptides, lipids, carbohydrates, amino acids, nucleic acids and others.

In one embodiment, the microbial growth by-product is ethyl acetate. In one embodiment, the ethyl acetate serves as a flavor enhancer in baked goods, for example, in sourdough bread. In one embodiment, the ethyl acetate is an antifungal agent.

In one embodiment, the microbial growth by-product is a biosurfactant. Biosurfactants are a structurally diverse group of surface-active substances produced by microorganisms. Biosurfactants are biodegradable and can be produced by selected organisms using renewable substrates. Most biosurfactant-producing organisms produce biosurfactants in response to the presence of a hydrocarbon source (e.g. oils, sugar, glycerol, etc.) in the growing media. Other media components such as concentration of iron can also affect biosurfactant production significantly.

Microbial biosurfactants are produced by a variety of microorganisms such as bacteria, fungi, and yeasts. Exemplary biosurfactant-producing microorganisms include Starmerella spp. (S. bombicola), Pseudomonas spp. (P. aeruginosa, P. putida, P. florescens, P. fragi, P. syringae); Flavobacterium spp.; Bacillus spp. (B. subtilis, B. pumillus, B. cereus, B. amyloliquefaciens, B. licheniformis); Wickerhamomyces spp. (e.g., W. anomalus). Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp.; as well as others listed herein.

Biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances and accumulate at interfaces, thus reducing interfacial tension and leading to the formation of aggregated micellar structures in solution. The ability of biosurfactants to form pores and destabilize certain cell membranes permits their use as antibacterial, antifungal, and hemolytic agents.

Biosurfactants according to the subject invention can include, for example, glycolipids, lipopeptides, flavolipids, phospholipids, fatty acid ester, and high-molecular-weight biopolymers such as lipoproteins, lipopolysaccharide-protein complexes, and/or polysaccharide-protein-fatty acid complexes.

In one embodiment, the biosurfactant is selected from glycolipids such as, for example, rhamnolipids (RLP), sophorolipids (SLP), cellobiose lipids, trehalose lipids and mannosylerythritol lipids (MEL). In one embodiment, the biosurfactant is selected from lipopeptides, such as, e.g., surfactin, iturin, fengycin, arthrofactin, amphisin, viscosin and/or lichenysin. In one embodiment, the biosurfactant is another type of amphiphilic molecule, such as, for example, esterified fatty acids, phospholipids (e.g., cardiolipins), and biopolymers, such as pullulan, emulsan, lipomanan, alasan, and/or liposan.

In preferred embodiments, the composition comprises a blend of one or more biosurfactants. Preferably, the composition comprises the biosurfactant blend at a concentration of about 0.001 to 0.5%, or about 0.01 to 0.1% by weight. In one embodiment, the biosurfactant blend comprises a glycolipid, a lipopeptide and/or a phospholipid

In one exemplary embodiment, the biopreservative composition comprises a sophorolipid at a concentration of 0.1% by weight. In one exemplary embodiment, the composition comprises a surfactin at a concentration of 0.01% by weight.

In one embodiment, the composition can comprise other microbial growth by-products and/or metabolites that can be useful for enhancing the quality of baked goods, including, for example, enzymes, biopolymers, solvents, acids or proteins.

In one embodiment, the biopreservative composition comprises the microbial growth by-products separated from the microorganisms that produced them. The growth by-products can be in a purified or unpurified form. Purification can be performed using known methods, for example, using a rotoevaporator, microfiltration, ultrafiltration, or chromatography.

The biopreservative composition can also comprise appropriate additives depending on its formulation and intended use, for example, buffering agents, carriers, other microbe-based compositions produced at the same or different facility, viscosity modifiers, biocides, emulsifying agents, lubricants, solubility controlling agents, pH adjusting agents, flavor enhancers, and stabilizers.

### Growth of Microbes

The subject invention provides methods for cultivation of microorganisms and production of microbial metabolites and/or other by-products of microbial growth. In one embodiment, the subject invention provides materials and methods for the production of biomass (e.g., viable cellular material), extracellular metabolites, residual nutrients and/or intracellular components.

The growth vessel used for growing microorganisms can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, isopropyl, propanol, butanol, pentanol, hexanol, isobutanol, and/or glycerol; fats and oils such as soybean oil, rice bran oil, canola oil, olive oil, corn oil, sesame oil, and/or linseed oil; etc. These carbon sources may be used independently or in a combination of two or more.

In one embodiment, the method comprises use of two carbon sources, one of which is a saturated oil selected from canola, vegetable, corn, coconut, olive, or any other oil suitable for use in, for example, cooking. In a specific embodiment, the saturated oil is 15% canola oil or discarded oil that has been used for cooking.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts can be included, such as potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride and/or sodium carbonate. These inorganic salts may be used independently or in a combination of two or more.

In some embodiments, the method for cultivation may further comprise adding additional acids and/or antimicrobials in the liquid medium before and/or during the cultivation process. Antimicrobial agents or antibiotics are used for protecting the culture against contamination. Additionally, antifoaming agents may also be added to prevent the formation and/or accumulation of foam when gas is produced during cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention provides methods of producing a microbial metabolite by cultivating a microbe strain of the subject invention under conditions appropriate for growth and production of the metabolite; and, optionally, purifying the metabolite. In a specific embodiment, the metabolite is a biosurfactant. The metabolite may also be, for example, ethanol, lactic acid, beta-glucan, proteins, amino acids, peptides, metabolic intermediates, polyunsaturated fatty acids, and lipids. The metabolite content produced by the method can be, for example, at least 20%, 30%, 40%, 50%, 60%, 70%, 80%, or 90%.

The biomass content of the fermentation medium may be, for example from 5 g/l to 180 g/l or more. In one embodiment, the solids content of the medium is from 10 g/l to 150 g/l.

The microbial growth by-product produced by microorganisms of interest may be retained in the microorganisms or secreted into the growth medium. In another embodiment, the method for producing microbial growth by-product may further comprise steps of concentrating and purifying the microbial growth by-product of interest. In a further embodiment, the medium may contain compounds that stabilize the activity of microbial growth by-product.

The method and equipment for cultivation of microorganisms and production of the microbial by-products can be performed in a batch, quasi-continuous, or continuous processes.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia or other microbial propagules. In this manner, a quasi-continuous system is created.

Advantageously, the methods of cultivation do not require complicated equipment or high energy consumption. The microorganisms of interest can be cultivated at small or large scale on site and utilized, even being still-mixed with their media. Similarly, the microbial metabolites can also be produced at large quantities at the site of need.

### Microbial Strains

The microorganisms useful according to the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microorganism is any yeast or fungus. Examples of yeast and fungus species suitable for use according to the current invention, include, but are not limited to, Acaulospora, Aspergillus, Aureobasidium (e.g., A. pullulans), Blakeslea, Candida (e.g., C. albicans, C. apicola), Debaryomyces (e.g., D. hansenii), Entomophthora, Fusarium, Hanseniaspora (e.g., H. uvarum), Hansenula, Issatchenkia, Kluyveromyces, Mortierella, Mucor (e.g., M piriformis), Penicillium, Phythium, Phycomyces, Pichia (e.g., P. anomala, P. guielliermondii, P. occidentalis, P. kudriavzevii), Pseudozyma (e.g., P. aphidis), Rhizopus, Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T. virens), Ustilago (e.g., U. maydis), Wickerhamomyces (e.g., W. anomalus), Williopsis, Zygosaccharomyces (e.g., Z. bailii).

In one embodiment, the microorganism is any yeast known as a “killer yeast.” As used herein, “killer yeast” means a strain of yeast characterized by its secretion of toxic proteins or glycoproteins, to which the strain itself is immune. The exotoxins secreted by killer yeasts are capable of killing other strains of yeast, fungi, or bacteria. Killer yeasts can include, but are not limited to, Wickerhamomyces, Pichia, Hansenula, Saccharomyces, Hanseniaspora, Ustilago Debaryomyces, Candida, Cryptococcus, Kluyveromyces, Torulopsis, Williopsis, Zygosaccharomyces and others.

In a specific embodiment, the microorganism is Wickerhamomyces anomalus. In certain embodiments, Wickerhamomyces anomalus can produce exo-β-1,3-glucanase, biosurfactants, as well as various other useful solvents, enzymes and metabolites, such as phytase, glycosidases, ethyl acetate, acetic acid, lactic acid, isopropyl alcohol, ethanol and phospholipids that resemble certain mammalian cardiolipins in structure.

These metabolites can be beneficial for a number of reasons. For example, exo-β-1,3-glucanase, as well as certain glycolipids and lipopeptides, can have antifungal properties. Additionally, ethyl acetate can improve the taste and texture of bread, and in some embodiments, can also have antifungal properties.

Furthermore, the phytase enzyme that can help improve the bioavailability of phosphorus from indigestible phosphates, thus improving the nutritional content of a baked good. Even further, the cardiolipin-like phospholipids that W. anomalus can produce may be helpful as a health supplement for subjects who have a metabolic or mitochondrial disorder caused by a cardiolipin deficiency.

In one embodiment, the microorganism is Saccharomyces cerevisiae, which is a common yeast used in baking as a leavening agent.

In one embodiment, the microorganism is Starmerella bombicola, which is an efficient producer of glycolipid biosurfactants. In one embodiment, the microbe is a strain of Pseudozyma aphidis, which is an effective producer of mannosylerythritol lipid biosurfactants.

In some embodiments, the microorganisms are bacteria, including Gram-positive and Gram-negative bacteria. The bacteria may be, for example Bacillus spp. (e.g., B. subtilis, B. licheniformis, B. firmus, B. laterosporus, B. megaterium, B. amyloliquefaciens, B. coagulans), Bacteroides spp., Clostridium spp., Faecalibacterium spp., Eubacterium spp., Ruminococcus spp., Peptococcus spp., Peptostreptococcus spp., Enterococcus spp., Bifidobacterium spp., Lactobacillus spp., Enterobacter spp., Klebsiella spp., and/or Escherichia spp.

In one embodiment, the microorganism is B. coagulans GBI-30 (BC30). In certain embodiments, the BC30 is in spore form.

In one embodiment, BC30 is a probiotic that contributes to the enhanced quality of baked goods by enhancing the nutritional value to a consumer through improved digestive and immune health. In certain embodiments, BC30 is a preferred probiotic for the present invention because it is capable of surviving the acidity of the stomach, thus allowing it to reach the intestines. BC30 contains a natural protective layer of proteins, which allows it to not only survive the harsh environment of the stomach, but also allows it to survive most manufacturing processes—including baking.

Other microbial strains can be used in accordance with the subject invention, including, for example, any other strains having high concentrations of mannoprotein and/or beta-glucan in their cell walls and/or that are capable of producing biosurfactants and other metabolites useful for preserving food.

### Preparation of Microbe-based Products

One microbe-based product of the subject invention is simply the fermentation medium containing the microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or dormant form, or the compositions may comprise combinations of active and dormant microorganisms.

In some embodiments, a growth by-product of the microorganism is extracted from the medium in which it was produced, and, optionally, purified.

The microbe-based products may be used without further stabilization, preservation, and storage. The microbes, growth by-products and/or medium resulting from the microbial growth can be removed from the growth vessel and transferred via, for example, piping for immediate use.

In other embodiments, the composition (microbes, medium, growth by-products, or combinations thereof) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

In certain embodiments, use of unpurified microbial growth by-products according to the subject invention can be superior to, for example, purified microbial metabolites alone, due to, for example, the advantageous properties of yeast cell walls. These properties include high concentrations of mannoprotein as a part of yeast cell wall's outer surface (mannoprotein is a highly effective bioemulsifier) and the presence of biopolymer beta-glucan (an emulsifier) in yeast cell walls. Additionally, the yeast fermentation product further can comprise biosurfactants and other metabolites (e.g., lactic acid, ethyl acetate, ethanol, phospholipids, etc.) in the culture.

In certain embodiments, upon harvesting the microbe-based composition from the growth vessels, the yeasts can be processed and formulated as, for example, active dry yeast, instant yeast, compressed yeast, cream yeast, rapid-rise yeast, and/or deactivated yeast, using known methods.

Additional components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Example of such additives include surfactants, emulsifying agents, lubricants, buffering agents, solubility controlling agents, pH adjusting agents, and stabilizers.

In one embodiment, the composition may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In one embodiment, additional components such as an aqueous preparation of a salt, such as sodium bicarbonate or carbonate, sodium sulfate, sodium phosphate, or sodium biphosphate, can be included in the formulation.

Advantageously, in accordance with the subject invention, the microbe-based product may comprise medium in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Methods

In certain embodiments, methods are provided for enhancing the quality of a baked good, wherein a biopreservative composition of the subject invention is baked into the baked good. The methods utilize components that are biodegradable and toxicologically safe, and can serve as replacements for potentially harmful additives and preservatives, such as, for example, sodium benzoate, potassium sorbate and calcium propionate.

Advantageously, the compositions and methods of the subject invention can be effective for preserving food and preventing food-borne illnesses without negatively altering the taste, smell, appearance, texture and/or nutritional value of food products. In fact, the compositions and methods can be used to enhance such properties, thus improving the overall experience and value of consuming the products.

In specific embodiments, the methods comprise forming a dough using, for example, flour, water or another liquid, and optionally, one or more of a sugar, salt, fat, oil, egg, milk or flavoring, wherein the biopreservative composition is incorporated into the dough. In certain embodiments, the biopreservative composition is added in place of, or in addition to, a fermenting agent and/or a leavening agent (e.g., standard baker's yeast).

The method can further comprise mixing, kneading, folding, proofing, fermenting, glazing, scoring, chilling, forming, topping, baking and/or processing the dough in any other way according to standard procedures and depending on what baked good is being produced.

In one embodiment, the composition comprises W. anomalus and/or S. cerevisiae. In one embodiment, the composition further comprises a probiotic, such as BC30.

In one embodiment, the composition comprises about 0.001% to 0.5% by weight of a biosurfactant blend comprising sophorolipids (e.g., 0.1%) and surfactin (e.g., 0.01%) and/or a phospholipid (e.g., 0.01% to 0.05%). In one embodiment, the composition further comprises ethyl acetate.

In some embodiments, the composition is mixed with water prior to incorporating it into the other ingredients of the dough.

The subject methods enhance the quality of baked goods by, for example, extending the consumable life, reducing the risk of illness or harm due to microbial contaminants (e.g., bread molds), as well as improving the taste, texture and/or nutritional value of the product, compared with products baked using traditional ingredients.

As used herein, the term “food product” refers to any substance, preparation, composition or object that is suitable for consumption, nutrition, oral hygiene or pleasure, and which are intended to be introduced into the human or animal oral cavity, to remain there for a certain period of time and then to either be swallowed or to be removed from the oral cavity again (e.g., chewing gum).

In preferred embodiments, the food product is a baked good, or a food product made of dough (e.g., comprising flour of wheat, maize, rice, oats, rye, legumes, nuts, seeds, or other cereal crops and water or another liquid) that requires leavening (e.g., by yeast, sodium bicarbonate, baking powder and/or cream of tartar) and is then baked in an oven (or another source of heat). Baked goods can include bread (e.g., a loaf, roll, muffin, biscuit, breadstick, dumpling, bun, pita, or naan), and pizza crusts, bagels, pretzels, doughnuts, cakes, cookies, pastries, pancakes, pasta, brownies, waffles, pies, tarts, puddings, and the like.

In some embodiments, the method prevents and/or controls undesirable microbial growth in and on the baked good. Thus, in addition to prolonging the consumable life of the baked good and preventing microbial alteration and/or decomposition thereof, the method can also be used to enhance the safety of the baked good for consumption, e.g., by preventing food poisoning or illness from molds and other pathogenic food-borne microorganisms.

The subject compositions and methods can be used to prevent and/or control the growth of undesirable fungi, bacteria (both Gram-negative and Gram-positive), mold, viruses and many other pests. Non-limiting examples of microbial agents that can cause the spoilage and/or contamination of fresh food products include bacteria, such as certain strains of Bacillus, Alicyclobacillus, Geobacillus, Lactobacillus, Proteus, Serratia, Klebsiella, Obesumbacterium, Campylobacter, Clostridrium, Erwinia, Salmonella, Staphylococcus, Shigella, Yersinia, Moraxella, Photobacterium, Thermoanaerobacterium, Desulfotomaculum, Pediococcus, Leuconostoc, Oenococcus, Acinetobacter, Leuconostoc, Psychrobacter, Pseudomonas, Alcaligenes, Serratia, Micrococcus, Flavobacterium, Proteus, Enterobacter, Streptococcus, Xanthomonas campestris, Listeria monocytogenes, Shewanella putrefaciens, Escherichia coli, and Vibrio cholerae;

viruses, such as mosaic virus, rotaviruses and hepatitis A;

parasites, such as tapeworms, Trichinella, Giardia lambda, and Entamoeba histolytica;

fungi, such as Zygosaccharomyces, Debaryomyces hansenii, Candida, and Dekkera/Brettanomyces; and

molds, such as Alternaria, Aspergillus, Byssochlamys, Botrytis, Cladosporium, Fusarium, Geotrichu, Manoscus, Monilia, Mortierella, Mucor, Neurospora, Oidium, Oosproa, Penicillium. In preferred embodiments, the method controls bread molds, e.g., Rhizopus stolonifera.

In some embodiments, the method results in the production of a baked good with unexpectedly enhanced taste and/or texture. For, example, in one embodiment, the subject method intensifies the “sour” flavor of sourdough bread. In another embodiment, the method increases the rise of bread dough, leading to improved texture through a lighter, fluffier, airier, and/or more elastic baked bread.

In some embodiments, the method results in the production of a baked good with enhanced nutritional value. For example, with the addition of a probiotic microorganism, such as, for example, BC30, the baked good can provide a consumer with health benefits such as improved digestion, reduced inflammation, balancing of the gut microbiome, and regulation of imbalances in lipid metabolism and the immune system.

Additionally, BC30 may out-compete other harmful microorganisms that cause infections or may have other deleterious effects, and can help in replenishing beneficial bacteria in the intestines for individuals who have an imbalanced gut microbiome due to illness and/or being prescribed antibiotics.

In some embodiments, the method can be used simultaneously with other standard methods of preservation. For example, the method can be used in combination with refrigeration, freezing and/or active packaging.

In some embodiments, the method can be used to reduce or eliminate the need for freezing or refrigerating baked goods to preserve them for longer than, for example, a week.

The methods can be used at any scale of food preparation, either by commercial or industrial bakers, or in a home kitchen, a local bakery or a restaurant.

In some embodiments, the subject invention provides an enhanced food product produced according to the subject methods. Preferably, the food product is a baked good. In a specific embodiment, the food product is a form of sourdough bread, e.g., a loaf, bun, boule, or roll.

### Local Production of Microbe-Based Products

In preferred embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The distributed microbe growth facilities can be located at the location where the microbe-based product will be used (e.g., a bakery). For example, the microbe growth facility may be less than 300, 250, 200, 150, 100, 75, 50, 25, 15, 10, 5, 3, or 1 mile from the location of use.

The microbe growth facilities of the subject invention produces fresh, microbe-based compositions, comprising the microbes themselves, microbial metabolites, and/or other components of the broth in which the microbes are grown. If desired, the compositions can have a high density of vegetative cells or propagules, or a mixture of vegetative cells and propagules.

Because the microbe-based product is generated locally, without resort to the microorganism stabilization, preservation, storage and transportation processes of conventional microbial production, a much higher density of bacteria cells and/or propagules can be generated, thereby requiring a smaller volume of the microbe-based product for use in the on-site application or which allows much higher density microbial applications where necessary to achieve the desired efficacy. Local generation of the microbe-based product also facilitates the inclusion of the growth broth in the product. The broth can contain agents produced during the fermentation that are particularly well-suited for local use.

Advantageously, the compositions can be tailored for use at a specified location. The microbe growth facilities provide manufacturing versatility by the ability to tailor the microbe-based products to improve synergies with destination geographies and harness the power of naturally-occurring local microorganisms and their metabolic by-products to improve oil production. Local microbes can be identified based on, for example, salt tolerance and ability to grow at high temperatures.

Advantageously, these microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated broth and metabolites in which the cells are originally grown.

The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

1. A composition for enhancing the quality of bread and baked goods, the composition comprising a Wickerhamomyces anomalus yeast and/or a microbial growth by-product of said yeast.

2. The composition of claim 1, wherein the growth by-product is ethyl acetate.

3. The composition of claim 1, wherein the growth by-product is a biosurfactant.

4. The composition of claim 3, wherein the biosurfactant is a sophorolipid, a surfactin and/or a phospholipid.

5. The composition of claim 1, further comprising a Saccharomyces cerevisiae yeast

6. (canceled)

7. The composition of claim 1, formulated as active dry yeast, instant yeast, compressed yeast, cream yeast, rapid-rise yeast, or deactivated yeast.

8. The composition of claim 1, further comprising a probiotic microorganism.

9. The composition of claim 8, wherein the probiotic is Bacillus coagulans GBI-30 (BC30).

10. A method for enhancing the quality of a baked good, the method comprising forming a dough using flour and water, and optionally, one or more of a sugar, salt, fat, oil, egg, milk and flavoring, and incorporating a biopreservative composition into the dough, wherein the composition is used in place of, or in addition to, a leavening agent, and wherein the biopreservative composition comprises a Wickerhamomyces anomalus yeast and/or a microbial growth by-product of said yeast.

11. The method of claim 10, further comprising mixing, kneading, folding, flavoring, proofing, fermenting, glazing, scoring, chilling, forming, topping, baking and/or processing the dough.

12. The method of claim 10, wherein the biopreservative composition is dissolved in water prior to being incorporated into the dough.

13. The method of claim 10, wherein the baked good is a bread in the form of a loaf, roll, muffin, biscuit, breadstick, bun, pita, or naan.

14. The method of claim 10, wherein the baked good is a pizza crust, bagel, pretzel, doughnut, cake, cookie, pastry, pancake, brownie, waffle, pie, tart, or pudding.

15. The method of claim 10, wherein the safety of the baked good for consumption is enhanced by preventing and/or controlling undesirable microbial growth in and on the baked good.

16. (canceled)

17. The method of claim 10, wherein the consumable life of the baked good is prolonged.

18. (canceled)

19. The method of claim 10, wherein the taste and/or texture of the baked good is improved.

20. The method of claim 19, wherein the baked good has an increased rise compared with baked goods baked with traditional ingredients.

21. The method of claim 10, further comprising incorporating a probiotic into the dough, wherein the nutritional value of the baked good is improved.

22. (canceled)

23. An enhanced food product produced according to a method of claim 10, the food product comprising a sourdough bread.

24. The sourdough bread of claim 23, said sourdough bread having an increased rise compared with bread baked using traditional methods.
